(19) United States
(12) Patent Application Publication
(10) Pub. No.: US 2022/0105029 A1
(43) Pub. Date: Apr. 7, 2022

(54) BIODEGRADABLE CHITOSAN MICRONEEDLE PATCH FOR TRANSDERMAL DELIVERY FOR LIVESTOCK PAIN MANAGEMENT

(71) Applicant: THE BOARD OF TRUSTEES OF THE UNIVERSITY OF ARKANSAS, Little Rock, AR (US)

(72) Inventors: Jorge L. Almodovar, Fayetteville, AR (US); Lauren Greenlee, Fayetteville, AR (US); Jeremy G. Powell, Lowell, AR (US); David Alfonso Castilla-Casadiego, Fayetteville, AR (US)

(21) Appl. No.: 17/496,515

(22) Filed: Oct. 7, 2021

Related U.S. Application Data
(60) Provisional application No. 63/088,783, filed on Oct. 7, 2020.

(51) Int. Cl. A61K 9/00; A61K 31/5415; A61K 47/36; A61M 37/00; B29C 41/00; B29C 41/02

(52) U.S. Cl. CPC A61K 9/0021; A61K 31/5415; A61K 47/36; A61M 37/0015; B29K 2105/0035; B29C 41/02; A61M 2037/0053; A61M 2037/0023; B29K 2005/00; B29C 41/003

(57) ABSTRACT

Disclosed herein is a microneedle array comprising a substrate and a plurality of microneedles extending therefrom, wherein the microneedles comprise a biodegradable polymer and an effective amount of an analgesic or an anti-inflammatory therapeutic agent. Methods of using and making the microneedle array are also disclosed.

### CROSS-REFERENCE TO RELATED APPLICATIONS

This application claims benefit of priority to U.S. Patent Application Ser. No. 63/088,783, filed Oct. 7, 2020, the contents of which is incorporated by reference in its entirety.

### FIELD OF THE INVENTION

The disclosed technology is generally directed to compositions and methods for drug delivery. More particularly the technology is directed to biodegradable chitosan microneedle patches for transdermal delivery of therapeutic cargo.

### BACKGROUND OF THE INVENTION

The term “animal welfare” refers to the relationships that people have with animals, specifically related to the duty of assuring that the animals under their care are treated compassionately and responsibly [1]. Nowadays, this term is being used increasingly by a significant percentage of the society including companies, consumers, transporters, veterinarians, scientists, and politicians [2]. Veterinarians and farmers have reported that if an animal is healthy they will deliver products with a promising quality [3]. Countries including Canada, USA, Australia, and Belgium have amply demonstrated interest in farm animal welfare, reporting concerns during routine animal management practices such as castration, dehorning, and tail docking in cattle [2]. In the United States, dairy producers have recognized that these practices are painful, but analgesia is rarely provided [2][4][5]. Hence, many livestock animals are not handled daily. A reported survey of cattle veterinarians showed that only 52.9% of surveyed veterinarians provided analgesia during the castration of cattle older than 6 months [6]. Consequently, inflicting and alleviating pain are mentioned continuously as key societal concerns for animal welfare [2]. Currently, the USA has been increasing public awareness and concern for the well-being of livestock animals [7][8][9]. Pain management for livestock has become an important issue for organizations like the American Veterinary Medical Association, which are now encouraging the use of pain relief during routine management practices in cattle [10]. Nevertheless, few pain medications exist commercially that are Food and Drug Administration (FDA)-approved for use in cattle.

The one FDA-approved pain management medication for livestock animals is flunixin meglumine, which is a non-steroidal anti-inflammatory drug (NSAID). A significant issue with flunixin meglumine is the degradation half-life, which is only 6.2 hours, and its administration by injection or topical application every 12 to 24 hours to maintain analgesia [11]. As consumer awareness of animal welfare increases, the need for US veterinarians and cattle producers to address animal pain mitigation also grows. With post-surgical pain sensation occurring over an extended period of time, pain control therapy is best applied over an extended period of time. However, no veterinary products with extended analgesic activity currently exist for livestock in the US [12].

### BRIEF SUMMARY OF THE INVENTION

Described herein are microneedle arrays and methods of making and using the same. In one aspect the microneedle array comprises a substrate and a plurality of microneedles extending therefrom, wherein the microneedles comprise a biodegradable polymer and an effective amount of an analgesic or an anti-inflammatory therapeutic agent. In some embodiments, the therapeutic agent is meloxicam and/or the biodegradable polymer is chitosan.

Another aspect of the invention is a method for treating a subject for pain or for pain management. The method may comprise administering the any of the microneedle arrays described herein to the epidermis of the subject, thereby piercing the stratum corneum of the subject. In some embodiments, microneedle array sustainably releases the therapeutic agent for at least one week.

Another aspect of the invention is a method for preparing the microneedle array. The method may comprise applying a microneedle composition, the microneedle composition comprising a biodegradable polymer and an effective amount of an analgesic or an anti-inflammatory therapeutic agent, to a mold, wherein the mold comprises a plurality recesses configured to prepare a plurality of microneedles; distributing the microneedle composition within the plurality of recesses; setting the microneedle array; and releasing the microneedle array from the mold.

These and other aspects of the inventions will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1B. Schematic illustrations of chitosan/meloxicam microneedle fabrication process for Example 1 (FIG. 1A) and Example 2 (FIG. 1B).

FIG. 2. Schematic view and transdermal drug delivery of a microneedle patch. A) Top view of microneedle patch and its dimensions. B) Slanted microneedle patch view of with 225 microneedles. C) Side view of microneedle and dimension of one microneedle. D) Zoom of microneedle with meloxicam incorporated. E) Schematic illustrations of transdermal delivery of meloxicam using chitosan/meloxicam microneedle patches.

FIG. 3. SEM pictures of chitosan and chitosan/meloxicam microneedles. A) Chitosan microneedles, C) Zoom of one chitosan microneedle. B) Chitosan/meloxicam microneedles, D) Zoom of one chitosan/meloxicam microneedle.

FIG. 4. FTIR spectrums. A) Chitosan microneedles, B) Chitosan/meloxicam microneedles, C) Pure chitosan, D) Pure meloxicam.

FIG. 5. Penetration testing. A) Macroscopy picture of microneedle patch, B) Microneedle patch and cow's ear cadaver skin. C) SEM picture of penetration in the skin, D) Penetration of one microneedle, E) Depth penetration in the skin.

FIG. 6. Meloxicam release profile: The meloxicam loading amount in the microneedles was 1 mg per patch.

FIG. 7. Microscopy characterizations demonstrated that microneedles with high concentration of drug are uniformly organized on the patch surface and preserve their morphological properties after the sterilization process using ethylene oxide gas. Macroscopy view. A) PDMS mold vs. microneedle patches. B) Zoom of microneedle patch surface. Microscopy view. C) 3D laser image of microneedle patch. D) 3D laser image of one microneedle.

FIG. 8. A) SEM images of non-sterile (i) and sterile (ii) multiple microneedles patch 3D laser image of microneedle patch. B) SEM images of one non-sterile (i) and sterile (ii) microneedle.

FIG. 9. Insertion study demonstrated that microneedle patches were capable of degrading in vivo in a cow's ear. Microneedle patch topography shown before in vivo insertion and after 7 days of in vivo insertion.

FIG. 10. In vitro drug release analysis reports that microneedles provided a sustained release with approximately 33.02% of the meloxicam released for 7 days. A) In vitro % cumulative drug release profile of meloxicam from microneedle patches, B) Schematic representation of drug release (i) Brust delivery, drug on microneedle patch surface, (ii) Slower delivery, drug encapsulated, (iii), linear delivery, drug on microneedle patch surface and encapsulated. Data are presented as the mean±standard deviation of n=3 samples.

### DETAILED DESCRIPTION OF THE INVENTION

Disclosed herein is a microneedle array wherein the microneedles comprise a biodegradable polymer and an effective amount of a therapeutic agent. As demonstrated in the Examples, a chitosan-based biodegradable polymeric microneedle patch for the delivery of meloxicam as a pain management drug approach for use in cattle can be prepared. The microneedles sustainably release therapeutic agent slowly over time that allows for therapeutic treatment over an extended period of time. This system of biodegradable microneedle patch will help to extend the use of the therapeutic agents, such as meloxicam, for pain management medication for livestock animal and other subjects.

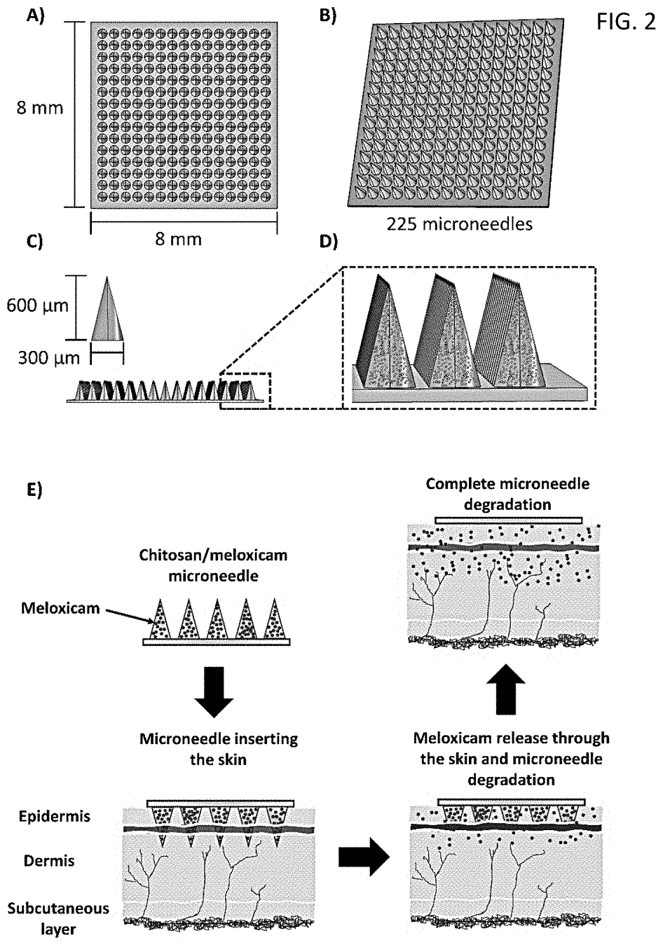

Microneedle (MN) arrays are minimally invasive devices that by-pass the stratum corneum (SC) barrier, thus accessing the skin microcirculation and achieving systemic delivery by the transdermal route. The intact SC provides the main barrier to transdermal delivery of exogenous substances, including drugs or other therapeutic agents. The SC is composed of corneocytes of hydrated keratin embedded in a lipid matrix of ceramides, fatty acids, cholesterol and its esters. These bilayers form regions of semicrystalline gel and liquid crystal domains. MNs pierce the epidermis, creating microscopic aqueous pores through which drugs diffuse to the dermal microcirculation. MNs are long enough to penetrate to the dermis but are typically short and narrow enough to avoid stimulation of dermal nerves or puncture of dermal blood vessels. (FIG. 2 (E))

MN arrays comprise a substrate and a plurality of MNs extending therefrom. The MN arrays may be formed in any suitable geometry for piercing the epidermis and allowing for transdermal delivery of therapeutic agents. The MN array may have a square geometry, as shown in FIG. 2 (A), but that need not be the case. Other two-dimensional geometries, such as circular, ovular, rectangular, and the like may be fabricated by those of skill in the art. The density of MNs may be from tens to thousands of microneedles per square centimeter. For example, the MN array may have up to 2000 MN cm⁻². (FIG. 2 (B)) The MN array may be sized or the density of MNs may be tailored as needed to control the amount of therapeutic agent to be delivered.

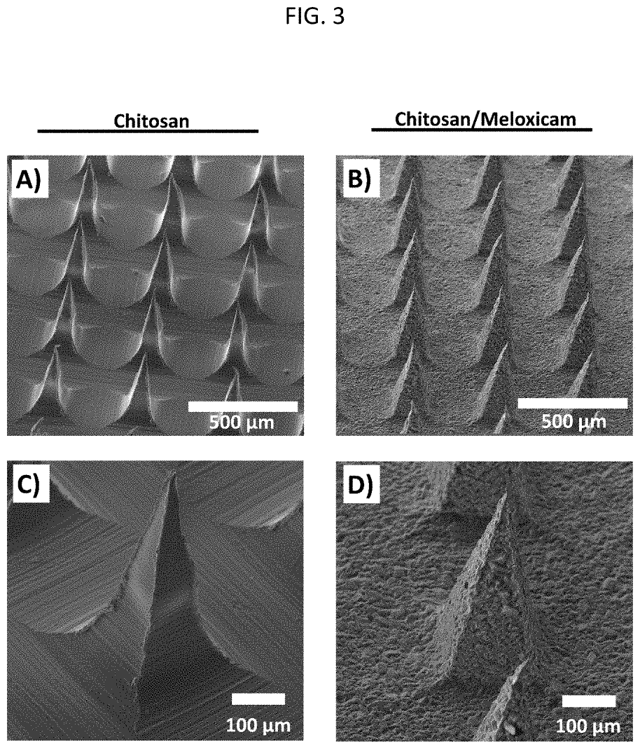

The MNs capable of penetrating the dermis and, preferably, avoid stimulation of dermal nerves or puncture dermal blood vessels. Suitably, MN may be between 50-900, 400-800, or 500-700 micron in height. For example, the MN may be 600 micron in height, as shown in FIGS. 2 (C) and (D). The MN may be formed in any suitable geometry for delivering the therapeutic agent. As shown in FIG. 3 (A)-(D), the MN may be pyramidal, but need not be. The aspect ratio of the base to height may be suitably selected depending on the application to ensure that the MN are robust enough to pierce the dermis. In the Examples, the aspect ratio of the base to height is 300 micron to 600 micron, but other ratios may also be used.

Therapeutic agents may be delivered by dissolving or degrading the MNs. MNs are made by micro-molding soluble matrices, comprising a biocompatible polymer or sugar and a therapeutic agent. The skin insertion of the array is followed by dissolution of the MN tips upon contact with skin interstitial fluid. The drug cargo is then sustainably released over time. Release can be sustained for days, weeks, or months depending on composition. The release kinetics of the drug depends upon the constituent polymer's dissolution rate. Therefore, controlled drug delivery is achievable by adjusting the polymeric composition of the MN array, or by modification of the MN fabrication process to control the size or number of MNs piercing the SC. Dissolving MNs present numerous advantages. A benefit is the low cost of polymeric materials and their facile fabrication by micro-molding. The use of water-soluble or biodegradable materials eliminates the potential risk of leaving biohazardous sharp waste in the skin. Moreover, safe MN disposal is facilitated, since the MN are, by definition, self-disabling.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. As used herein, “therapeutic agent” is any substance that may treat a subject. Therapeutic agents for use of the MN arrays may be selected for pain management or to treat pain. In some embodiments, the therapeutic agent is an analgesic or an anti-inflammatory agent. Suitably, the therapeutic agent is a nonsteroidal anti-inflammatory drug (NSAID) such as meloxicam or flunixin meglumine.

As used herein, a “subject” may be interchangeable with “patient” or “individual” and means an animal, which may be a human or non-human animal, such as livestock, in need of treatment. In some embodiments, the subject is a bovine, equine, ovine, caprine, porcine, or the like. A “subject in need of treatment” may include a subject having pain or in need of pain management.

As used herein the term “effective amount” refers to the amount or dose of the therapeutic agent, upon single or multiple dose administration to the subject, which provides the desired effect. The disclosed methods may include administering an effective amount of the therapeutic agent. An effective amount can be readily determined by the attending diagnostician, as one skilled in the art, by the use of known techniques and by observing results obtained under analogous circumstances. In determining the effective amount or dose of the therapeutic agent administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

In some embodiments, the effective amount of the therapeutic agent may be determined by the absolute amount of therapeutic agent present in the MN array. The Examples demonstrate the preparation of MN array having 50 and 125 mg of therapeutic agent but other amounts of a therapeutic agent may be used.

In some embodiments, the effective amount of the therapeutic agent may be determined by the amount of therapeutic agent per the area of the MN array. The Examples demonstrate the preparation of MN array having 50 and 125 mg of therapeutic agent per 64 mm² but the amount of a therapeutic agent and/or the area of the MN array may be varied.

Meloxicam is a NSAID possessing a long half-life of 28 hours. Currently, meloxicam is FDA-approved and routinely prescribed for pain mitigation in other veterinary species (i.e., dogs and cats). Meloxicam is approved as an analgesic for cattle in the European Union and Canada, where it is indicated for the alleviation of pain. Administration of meloxicam results in systemic analgesia and reduced inflammation by reducing the synthesis of inflammatory prostaglandin by inhibition of cyclooxygenase enzymes. Meloxicam is administered to animals orally through suspension or tablet form. Such administration is not amenable for livestock administration. Taking medications orally is not the best technique for treating problems or diseases in animals for several reasons. First, if the administration is done quickly, it can cause undesirable effects on the animal. For example, once oral medication is delivered to the animal, it cannot be withdrawn from the patient's bloodstream. Second, the oral administration of medications cannot be applied to animals that have undergone surgery on the stomach or intestine. Likewise, it cannot be administered to animals with vomiting or having unconsciousness problems because the animal may present problems with swallowing the medicine. Additionally, if the animal is anesthetized, there is a high risk of aspiration. Finally, this is a procedure that must be carried out more than once a day, which can cause some stress in the animal. The present technology is a superior approach for pain management in livestock via delivery of therapeutic agents via MN arrays. As demonstrated in the Examples, the MN arrays may be loaded with meloxicam, and optionally additional therapeutic agents, that will be released in the skin and delivered to the site of action by capillaries or lymphatic networks. As demonstrated in the Examples, the present technology allows for sustainable release over the period of days, weeks, or even months.

MN arrays of the present invention may be fabricated by molding a MN array with a MN composition. The MN composition comprises a biodegradable polymer and an effective amount of a therapeutic agent. The MN composition may further comprise a solvent or carrier, such as acetic acid, that allows for the preparation of the MN arrays. Suitably, the MN composition comprises an analgesic or an anti-inflammatory therapeutic agent, such as meloxicam or flunixin meglumine, and a biodegradable polymer such as chitosan, starch, dextran, or cellulose. The MN composition may be a liquid, solution, emulsion, or other flowable material capable of filling the mold's recesses.

In some embodiments, the biodegradable polymer is chitosan. Chitosan is a naturally biodegradable biopolymer. The rate of degradation may depend on a number of different factors, including, without limitation, molecular weight, deacetylation degree, polydispersity, purity level, and moisture content. Those of skill in the art may select the properties of the biodegradable polymer to control the rate of degradation. Chitosan is degraded in vivo by lysozymes or through the process of enzymatic transformation to basic, non-toxic components, such as oligosaccharides that are then excreted or incorporated to glycosaminoglycans and glycoproteins. Once the chitosan degradation is started into skin layers, the therapeutic agent will go into systemic circulation through the blood vessels until it reaches the targeted site of action to provide a therapeutic response.

To fabricate the MN array, the MN composition is applied to a mold. The mold comprises recesses that are suitably configured to provide the desired physical form for the MN array. Accordingly, the mold may be tailored to provide the desired geometry, surface area, MN height, MN shape, and so forth depending on the subject. The mold may be prepared from any suitable substance capable of providing the desired physical form for the MN array, and in some instances may be composed of polydimethylsiloxane (PDMS) or other suitable polymer or elastomer. The method may further comprise distributing the MN composition within the plurality of recesses in order to provide more uniform MN or minimize trapped gases that can result in deformed MNs. The MN composition may be distributed by centrifuging the MN composition within the mold, such as demonstrated in the Examples. Other methods may also be used to distribute the MN composition such as applying a force to the MN composition to force the composition into the mold's recesses via stamping or the like. Depending on the methods chosen for applying and distributing the MN composition, the application and distribution steps may be repeated one or more times until the recesses of the mold are suitably filled with MN composition. A substrate composition may be applied to provide a backing that facilities handling and administration of the MNs. In some embodiments, the MN composition and the substrate composition may be the same. In other embodiments, the MN composition and the substrate composition may be different. For example, the substrate material may comprise the biodegradable polymer without the therapeutic agent, but other materials may also be used. Optionally, the substrate composition may also be distributed over the MN composition to ensure uniformity. When employed, the substrate composition may be similarly distributed as the MN composition.

The MN array may be set. Suitably, setting results in the MN or substrate composition transforming from a flowable material to a solid material or other suitable material having the physical characteristics, such as strength or rigidity, that allow it to pierce the SC. The MN array may be set in any number of ways depending on the biodegradable polymer selected. For example, the MN or substrate composition may be dried or undergo a chemical reaction. In some embodiments, the substrate material is applied prior to setting the MNs, such as further described in Example 1. In other embodiments, the substrate material is applied after setting the MNs.

Finally, the MN array is released from the mold by peeling the microneedle array or any other suitable method.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Solution Preparation

Chitosan solution was prepared by dissolving chitosan powder in 10% (v/v) of acetic acid in water at a final concentration of 10% w/v. One (1) gram of chitosan was added to 10 mL of acetic acid at the different concentrations evaluated. For decreasing the dissolution time, the solution was placed in a heating plate set at 70° C. for 3 h. Once the chitosan solution was prepared, the chitosan/meloxicam solution consisted of adding and mixing 50 mg of meloxicam in one milliliter of chitosan solution, which was used to build the first layer of the patch. The initial experimental design for this formulation included 3%, 7%, 10%, 30%, 50%, 70%, and 90% (v/v) of acetic acid to prepare the solution. However, percentages under 10% (v/v) of acetic acid resulted in a highly viscous solution, which was not useful to prepare the microneedle patch. The patches were prepared using 10% (v/v) of acetic acid as solvent for the chitosan solution because it produced useful the microneedle patches.

### Preparation of Microneedle Patch (Example 1)

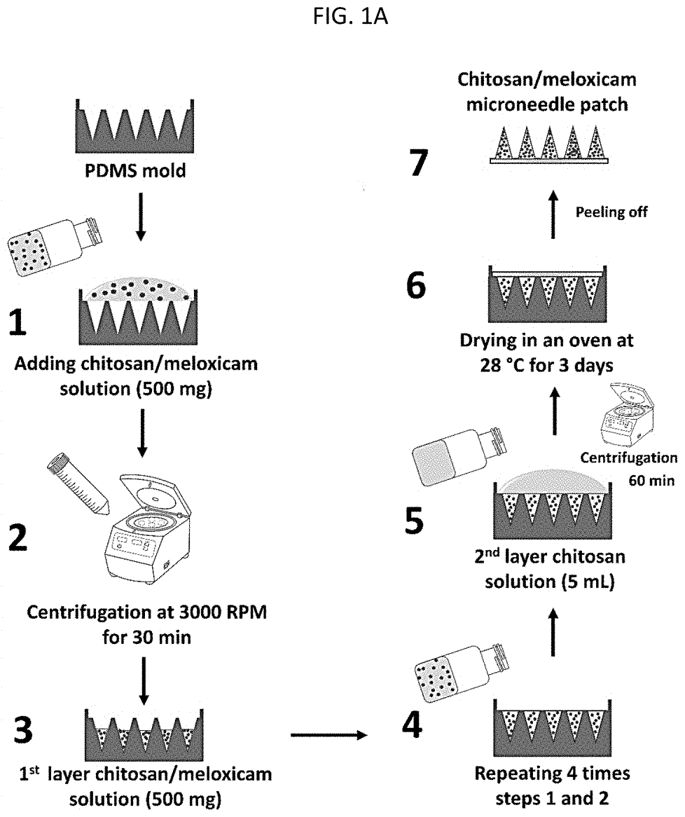

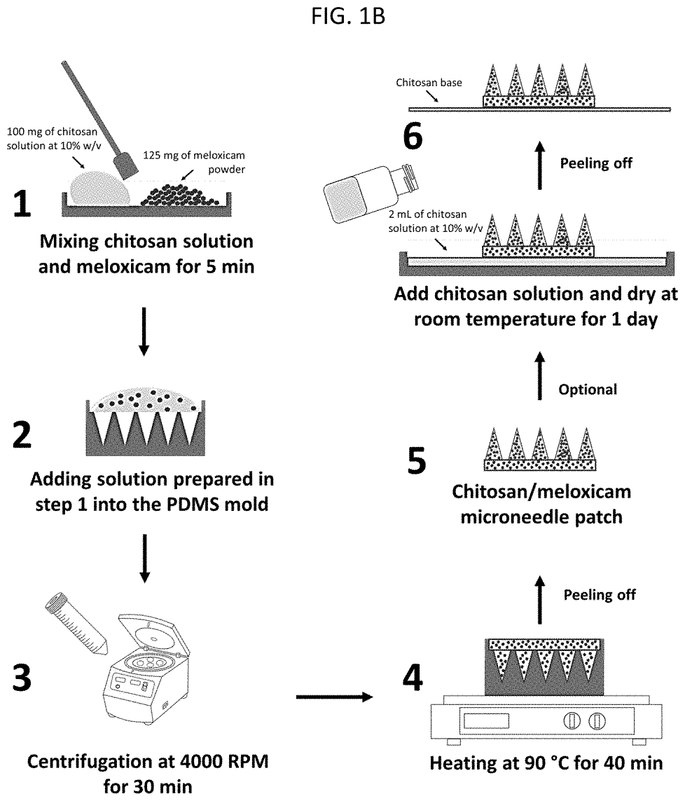

The microneedle patch was prepared by placing chitosan/meloxicam solution (prepared by: 1 gr of chitosan and 50 mg of meloxicam in 10 mL of acetic acid at 10%) on a PDMS mold with the following characteristics: Patch size: 8*8 mm, needle height: 600 needle base: 300 and an array size: 15*15 (225 microneedle/patch). (See FIG. 1A and FIG. 2 A-D) The process of fabricating the chitosan/meloxicam microneedle patch was conducted by adding first 500 mg of the solution onto the mold to create a monolayer (See FIG. 1A—step 1). After that the mold was put into a 50 mL centrifuge tube with flat bottom, this was centrifuged at 3000 RPM for 30 mins (See FIG. 1A—step 2 and 3). Then, steps 1 and 2 were repeated four times until it completed 120 mins of centrifuging (See FIG. 1A—step 4). Later, a second layer of chitosan solution was added. 5 mL of chitosan solution (prepared by: 1 gr of chitosan in 10 mL of acetic acid at 10%) were added into the 50 mL tube containing the mold with the chitosan/meloxicam solution (See FIG. 1A—step 5). Then again this was centrifuged at 3000 RPM for 60 mins. Finally, the mold into the 50 ml tube was put into an oven to dry without the cap at 28° C. for 3 days (See FIG. 1A—step 6). The microneedle patch was gently removed from the tube with tweezers (See FIG. 1A—step 7).

### Preparation of Microneedle Patch (Example 2)

The process of fabricating the microneedle patch is illustrated in FIG. 1B. The process began by of mixing 100 mg of chitosan solution with 125 mg of meloxicam (Molecular weight: 351.40 g/mol) purchased from Millipore Sigma (cat. no. PHR1799) for approximately 5 minutes and adding the homogenous mixture onto a (See FIG. 1B—step 1 and 2) polydimethylsiloxane (PDMS; Sylgard 184) mold purchased from Micropoint Technologies Pte, Ltd., Singapore (cat. no. ST-05). The dimensions of the mold were as follows: size 8 mm×8 mm, needle height 600 μm, needle base 300 μm, pitch 250 μm, and array size 15×15 (225 microneedles/patch). After that, the mold was put into a 50 mL centrifuge tube with a flat bottom and was centrifuged at 4000 RPM for 90 min using a Hettich ROTOFIX 32 A Cell Culture Centrifuge from VWR (cat. no. 10813-152) (See FIG. 1B—step 3). Then, the mold was placed on a hot plate to dry at 50° C. for 40 minutes; the mold surface was directly exposed to the hot plate surface, as shown in FIG. 1B—step 4. The microneedle patch in the mold was cooled at −20° C. for 5 minutes and gently removed from the mold with tweezers (See FIG. 1B—step 5). An optional step to generate a chitosan base can be added by spreading 2 mL of chitosan solution onto a flat surface, placing the microneedle patch at the center of the surface, and drying at room temperature for 1 day (See FIG. 1B—step 6). Microneedle patches without meloxicam (chitosan microneedle patches) were used as a control for the in vivo studies.

Example 2 increases the amount of drug from 50 mg/patch (Example 1) to 125 mg/patch. This methodology may prepare microneedle arrays with dimensions of: size 8 mm×8 mm, needle height 600 needle base 300 and array size 15×15 (225 microneedles/patch). This methodology is capable of producing 1 patch every 2 hours compared to 3 days. In addition, the amount of chitosan solution is reduced from 6 mL to approximately 2.1 mL if a base of chitosan is required.

Example 2 reduces the use of multiple patches per cattle and extra stress to the animal because this formulation provides a greater drug concentration of 2.5 times. Results on the optimization of increasing the concertation drug in the patch revealed that by using a mold with the dimensions as follows: size 8 mm×8 mm, needle height 600 needle base 300 pitch 250 and array size 15×15 (225 microneedles/patch), it is possible to fabricate patches with the capacity of release of a maximum of 125 mg of meloxicam. Patches with major amounts of the drug more than 125 mg resulted in defective patches, presenting several broken microneedles and a breakable surface with insufficient capacity to penetrate the skin.

The chitosan solution into the patch was reduced. The new preparation allowed the reduction of using 10 mL of chitosan solution at 10% (v/v) of acetic acid to 0.344 mL to prepare a single patch. The process of fabricating the chitosan/meloxicam microneedle patch consisted of mixing 100 mg of chitosan solution with 125 mg of meloxicam. This reduces the exposure of the animal to acetic acid during the drug release treatment.

### Sterilization of Microneedle Patches

Microneedle patches may be sterilized. In the Examples that follow, the patches were sterilized using ethylene oxide gas at room temperature for 24 hr.

### Morphology of the Microneedle Patches

Scanning electron microscopy (SEM) was used to characterize the morphology and distribution of microneedles on the chitosan and chitosan/meloxicam patch (FIG. 3) prepared by Example 1. FIG. 3 (A) and FIG. 3 (B) demonstrate that we can fabricate a chitosan and chitosan/meloxicam microneedle patch with a consistent distribution of microneedles and dimensions. FIG. 3(C) shows how one microneedle looks. This figure shows that the length of base of the microneedle is approximately 300 μm and the microneedle height is 600 Physically, the microneedle looks with a smooth and homogeneous top surface. On the other hand, FIG. 3 (D) shows that the chitosan/meloxicam microneedles have a consistent rough surface. This is related to meloxicam not dissolving in acids, meloxicam dissolves completely in few solvents such as, dimethyl sulfoxide (DMSO)[22].

### Chemical Composition of the Microneedle Patches

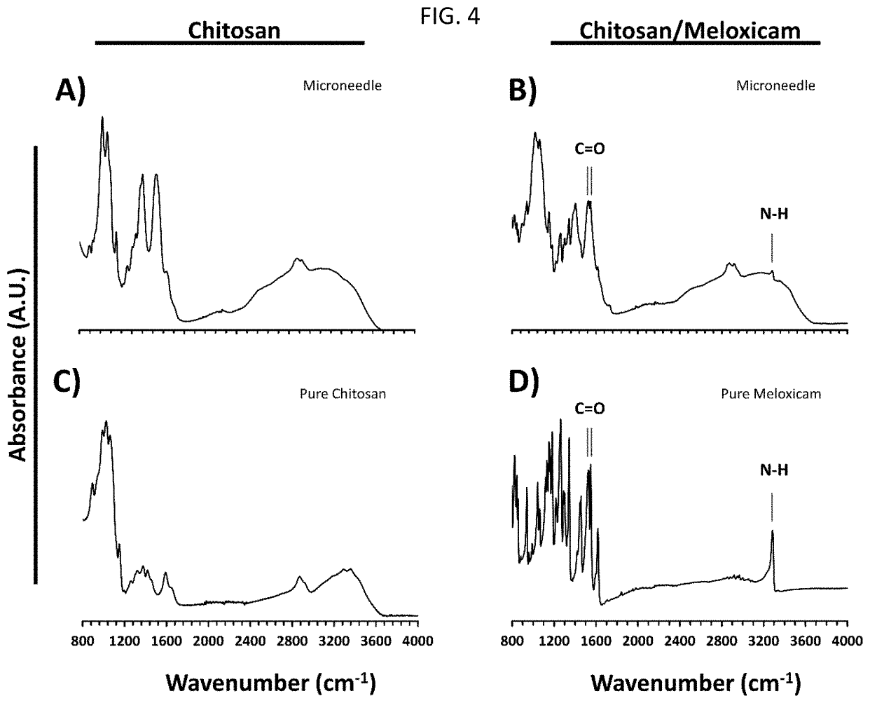

Fourier transform infrared spectroscopy (FTIR) was used to assess the chemical composition of chitosan and chitosan/meloxicam patches prepared by Example 1. FIG. 4 shows FTIR spectrums of the microneedle patches, pure chitosan, and pure meloxicam. Analyzing the FTIR spectra of pure chitosan in FIG. 4 (C), it is possible to observe that the characteristic absorption peaks of chitosan are shown at 3435 cm⁻¹ (—OH bond), 2922 cm⁻¹ (C—H stretch), 1656 cm⁻¹ (NH₂ deformation, amide I), 1603 cm⁻¹ (N—H, N-acetylated residues, amide II band), 1160 cm⁻¹ (bridge —O— stretch), 1085 cm⁻¹ (C—O stretch, secondary hydroxyl group), and 1030 cm⁻¹ (C—O stretch, primary hydroxyl group), as reported in the literature [23][24][25].

Comparing the FTIR spectrum for chitosan microneedle shown in FIG. 4 (A) with the pure chitosan spectrum in FIG. 4 (C), it is possible to observe that chitosan microneedle patch spectrum exhibits the same characteristic bands of chitosan. This validates that acid acetic does not alter the chemical composition of the chitosan as we demonstrated using other important polymers such as collagen [26][27].

Meloxicam FTIR spectrum is observed in FIG. 4 (D). This figure shows that the characteristic absorption peaks of meloxicam are shown at 3310 cm⁻¹ amine N—H stretch, 3201 cm⁻¹(O—H stretch), 2850 cm⁻¹ (aliphatic C—H stretch, 2905 cm⁻¹ (aromatic C—H stretch), 1560 cm⁻¹ (C═O stretch), 1340 cm⁻¹ (aromatic C═C stretch), 1190 cm⁻¹ (aromatic C═C stretch), as reported in the literature [28]. FIG. 4 (B) shows FTIR spectrum for chitosan/meloxicam microneedle, which presents the characteristic absorption peaks of pure chitosan and meloxicam, as described above. Analyzing the chitosan/meloxicam microneedle spectrum (FIG. 4 (B)), it is possible to observe that most of the characteristic bands of chitosan/meloxicam microneedle are located on the absorptions from 800 cm⁻¹ to 1600 cm⁻¹, similarly to pure chitosan and meloxicam (FIGS. 4 (B) and (D)). Clearly, chitosan/meloxicam microneedle spectrum shows the characteristic peaks of chitosan at 1160 cm⁻¹ (bridge —O— stretch), 1085 cm⁻¹ (C—O stretch, secondary hydroxyl group), and 1030 cm⁻¹ (C—O stretch, primary hydroxyl group). Likewise, the chitosan/meloxicam microneedle spectrum also presents the characteristic peaks of meloxicam. Notably, one of the most relevant peaks of meloxicam at 3310 cm⁻¹ (amine N—H stretch) is present on the chitosan/meloxicam microneedle spectrum (See label on FIGS. 4 (B) and (D)). Alike, it is possible to observe two peaks on both spectrums at 1560 cm⁻¹ which represents (C═O stretch) (See label on FIGS. 4 (B) and (D)). Hence, these results confirm that the chitosan/meloxicam microneedle patch contains meloxicam incorporated into chitosan microneedle.

### In Vitro Imaging of Microneedle Insertion in Cow's Ear Cadaver Skin

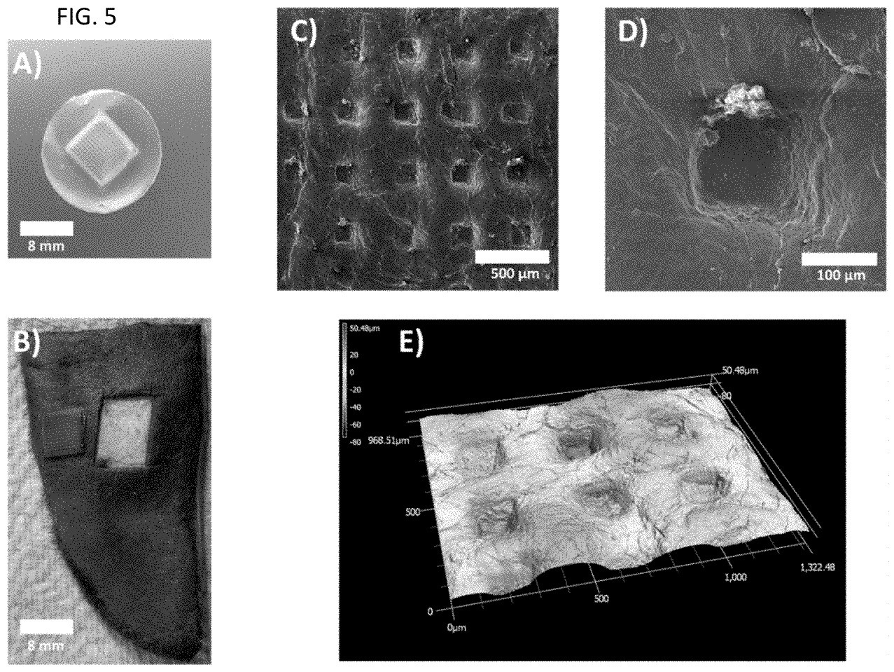

To evaluate the insertion capability of microneedles in vitro we inserted a microneedle patch prepared by Example 1 in cow's ear cadaver skin. FIG. 5 (A) shows a macroscopy picture of the microneedle patch used. This patch has 225 microneedles uniformly organized on an area of 8*8 mm with a distribution of 15*15 microneedle as was described above. FIG. 5 (B) shows the section of cow's ear cadaver skin where the microneedle patch was inserted. FIGS. 5 (C), (D), and (E) demonstrate that the penetration into the skin was effective. Using a SEM, FIGS. 5 (C) and 5 (D) show that the penetration on the skin is uniform because FIG. 5 (C) shows that every single microneedle is penetrating the skin keeping the distribution of the microneedle on the surface patch. FIG. 5 (D) shows the penetration of one single microneedle, demonstrating that the hole has approximately an area of 200*200 μm on the dry skin. Using a 3D laser microscope, it was possible to measure the penetration in the dry skin. The results show a depth penetration in the dry skin of approximately 78±1 Thus, these results indicate that the microneedle patch has capability of penetrating the skin, which is essential for the transdermal drug delivery in the cattle.

In Vitro Meloxicam Release from Microneedle Patch

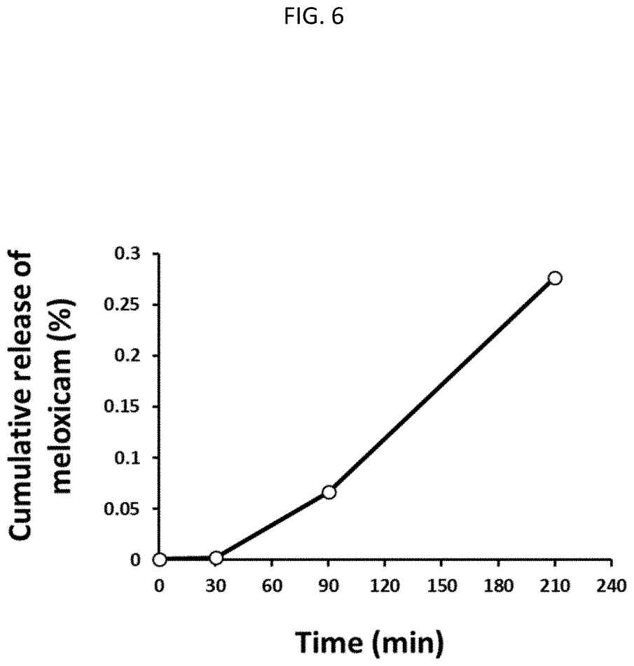

High-performance liquid chromatography (HPLC) was used to evaluate meloxicam release from the microneedle patch prepared by Example 1. The microneedle patch was placed into ultrapure water (10 mL), and samples were taken at 30, 60, 90, and 210 mins. FIG. 6 shows meloxicam is being slowly released. The sample taken at 210 mins reported a cumulative release of meloxicam of only 0.3%. Being a promising finding since the microneedle system helps to regulate and prolong the dosing time of meloxicam. Therefore, using the microneedle patch is possible to extend the use of the meloxicam how pain management medication for livestock animals and present a favorable alternative to eliminate the laborious and continuous orally administration during the pain release time.

### Morphology of Microneedle Patches

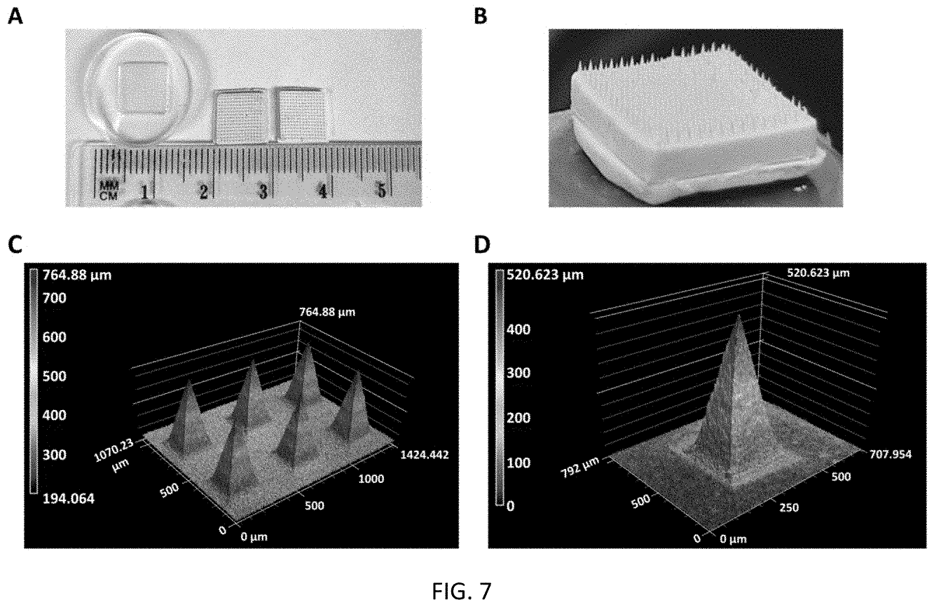

FIG. 7 shows the morphology of chitosan/meloxicam microneedle patch prepared by Example 2. The new high concentrated microneedle patch demonstrated to faithfully conserve the dimensions of the PDMS mold, as shown in FIG. 7 (A), which presents the type of mold (8 mm×8 mm) used during the fabrication and two microneedle patches. FIGS. 7 (C) and (D) confirm that the height and base length of the microneedles are approximately 600 μm and 300 μm, respectively, and a pitch of 250 μm, matching the dimensions of the mold used. FIG. 7 (B) shows the final aspect of the microneedle patch, which shows a patch surface roughness that is due to the presence of meloxicam nanoparticles, which is not soluble in acetic acid. This new formulation confirms the production of microneedle patches with low amount of the chitosan solution and insignificant traces of acetic acid. However, the amount of chitosan found in the patch was sufficient to provide support and mold the drug as a uniformly organized surface of microneedles.

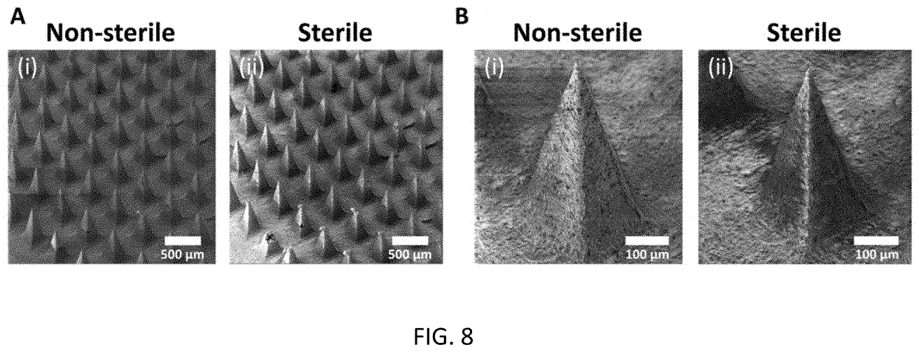

FIG. 8 shows the topography of the non-sterile and sterile chitosan/meloxicam microneedle patch prepared by Example 2. This result shows that the topographic characteristics of sterile patches are similar to non-sterile patches, which indicates that the sterilization process does not generate drastic damage to the surface. Hence, the MN patches prepared by Example 2 avoid the excessive use the chitosan solution. This eliminates or reduces an undesired response from the animal due to acetic acid being present in the patch. MN patches prepared by Example 2 promotes the optimization of microneedle patches production in short time, and amply increased the dose to be released, which permits the use of few microneedles patches to supply the required dose per cattle.

### In Vivo Degradation Capability Analysis of Microneedle Patch

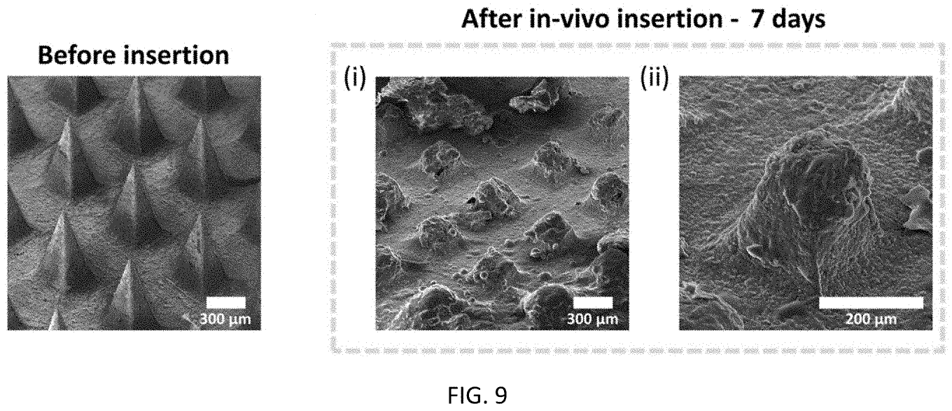

FIG. 9 demonstrates that the in vivo degradation of the microneedles administered to a cow's ear. Using SEM, morphological changes of the microneedles before and after in vivo insertion were assessed, as shown in FIG. 9 (right and left), respectively. After 7 days of insertion, results revealed that microneedles obtained more than 50% of degradation. FIG. 9 (i) shows that microneedles present an apparent homogeneous or uniform degradation, showing MNs having a height of less than 300 μm and rounded tips, to direct contact with the epidermis and dermis layers of the calf's ear skin. FIG. 9 (ii) shows the degradation of one single microneedle, demonstrating that the microneedle has approximately a height less than 300 μm from the original height of 600 μm. These results indicate that the microneedle patch has the capability to degrade in vivo when penetrating the epidermis and dermis layer of the skin. Additionally, at the area where the patch was inserted, there was no indication of abnormal tissue, swelling, or inflammation.

### In Vitro Drug Release Analysis

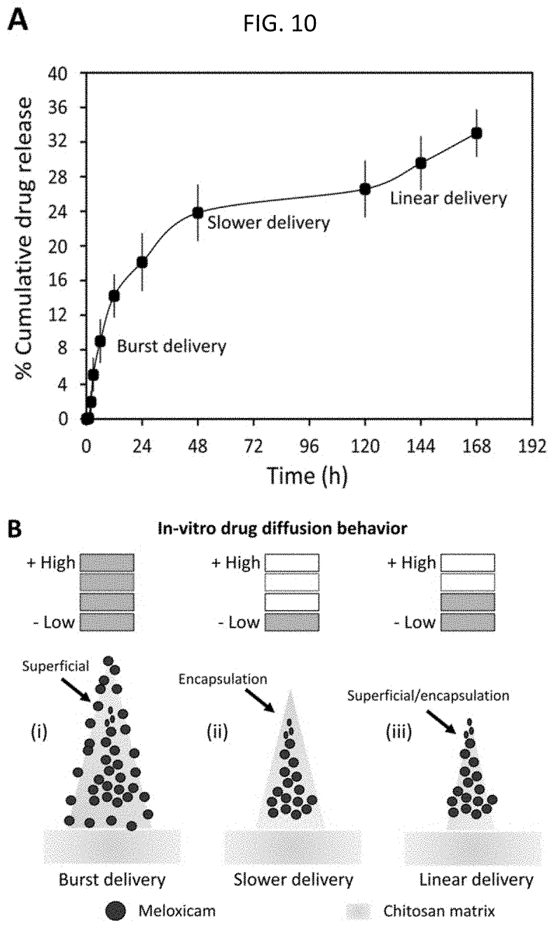

The release of meloxicam from microneedles is described by a triphasic pattern, which consisted of an initial rapid release during the first 2 days followed by a slower release within 2 to 5 days, and finally a linear release behavior (FIG. 10 (A)). The initial burst delivery could be associated to the immediate release of meloxicam on the microneedle surface (See FIG. 10 ((B(i)), which are initially and directly exposed to the DPBS dissolvent used for the in vitro drug release analysis. The roughness surface of the microneedle patch is due to the presence of meloxicam on the surface of the patch, which is not soluble in acetic acid. After 2 days, the patch showed a slower and constant release for three days, which could be attributed to meloxicam that is completely encapsulated in the chitosan matrix creating a barrier that prevents the rapid release of the drug (See FIG. 10 (B(ii)). Subsequently, after 5 days a linear trend was observed, which could be related to the proportional ratio of chitosan solution and meloxicam in the microneedle patch (See FIG. 10B (B(iii)). As shown in FIG. 10 (A), the microneedles provided sustained release with approximately 33.02% of the meloxicam present in one patch, which represents 41.28 mg of meloxicam released per patch in 7 days. These results demonstrated that the microneedle patch by itself without no modification to control degradation has the capability of providing an in vitro drug release for more than 7 days.

### REFERENCES

[1] C. J. Hewson, What is animal welfare? Common definitions and their practical consequences, Can. Vet. J. 44 (2003) 496.
[2] S. T. Millman, Behavioral responses of cattle to pain and implications for diagnosis, management, and animal welfare, Vet. Clin. Food Anim. Pract. 29 (2013) 47-58.
[3] A. Rauch, Jeff S. Sharp, Ohioans' attitudes about animal welfare, Soc. Responsib. Initiat. Dep. Hum. Community Resour. Dev. Ohio State Univ. (2005).
[4] F. G. H. Hoe, P. L. Ruegg, Opinions and practices of Wisconsin dairy producers about biosecurity and animal well-being, J. Dairy Sci. 89 (2006) 2297-2308.
[5] L. J. Misch, T. F. Duffield, S. T. Millman, K. D. Lissemore, An investigation into the practices of dairy producers and veterinarians in dehorning dairy calves in Ontario, Can. Vet. J. 48 (2007) 1249-1254.
[6] V. R. Fajt, S. A. Wagner, Bo Norby, Analgesic Drug Administration and Attitudes about Analgesia in Cattle among Bovine Practitioners in the United States, J. Am. Vet. Med. Assoc. 238 (2011) 755-767.
[7] L. Anil, S. S. Anil, J. Deen, Pain detection and amelioration in animals on the farm: issues and options, J. Appl. Anim. Welf Sci. 8 (2005) 261-278.
[8] D. R. Deemer, L. M. Lobao., Public Concern with Farm-Animal Welfare: Religion, Politics, and Human Disadvantage in the Food Sector, Rural Sociol. 76 (2011) 167-196.
[9] D. Weary, 218 Understanding and Responding to Public Concerns Regarding Farm Animal Welfare, J. Anim. Sci. 96 (2018) 5.
[10] American Veterinary Medical Association, Welfare Implications of Castration of Cattle, (n.d.). https://www.avma.org/KB/Resources/LiteratureReviews/Pages/castrationcattle-bgnd.aspx (accessed Sep. 20, 2002).
[11] D. E. Anderson, M. A. Edmondson, Prevention and Management of Surgical Pain in Cattle, Vet. Clin. Food Anim. Pract. 29 (2013) 157-184.
[12] M. Vivancos, J. Barker, S. Engbers, C. Fischer, J. Frederick, H. Friedt, J. M. Rybicka, T. Stastny, H. Banse, Alastair E. Cribb, Pharmacokinetics and bioequivalence of 2 meloxicam oral dosage formulations in healthy adult horses, Can. Vet. J. 56 (2015) 730-736.
[13] J. F. Coetzee, B. KuKanich, R. Mosher, P. S. Allen, Pharmacokinetics of Intravenous and Oral Meloxicam in Ruminant Calves, Vet Ther. 10 (2009) E1-E8.
[14] J. F. Coetzee, Review of Pain Assessment Techniques and Pharmacological Approaches to Pain Relief after Bovine Castration: Practical Implications for Cattle Production within the United States, Appl. Anim. Behav. Sci. 135 (2011) 92-213.
[15] E. A. Ochroch, I. A. Mardini, A. Gottschalk, What is the role of NSAIDs in pre-emptive analgesia?, Drugs. 63 (2003) 2709-2723.
[16] L. Fraccaro, E., Coetzee, J. F., Odore, R., Edwards-Callaway, L. N., Kukanich, B., Badino, P., Bertolotti, L., Glynn, H., Dockweiler, J., Allen, K. and Bergamasco, A study to compare circulating flunixin, meloxicam and gabapentin concentrations with prostaglandin E2 levels in calves undergoing dehorning, Res. Vet. Sci. 95 (2013) 204-211.
[17] Y. Ye, J. Yu, D. Wen, A. R. Kahkoska, Z. Gu, Polymeric microneedles for transdermal protein delivery, Adv. Drug Deliv. Rev. 127 (2018) 106-118.
[18] E. Larraneta, R. E. Lutton, A. D. Woolfson, R. F. Donnelly, Microneedle arrays as transdermal and intradermal drug delivery systems: Materials science, manufacture and commercial development, Mater. Sci. Eng. R Reports. 104 (2016) 1-32.
[19] E. Chen, M.-C.; Ling, M.-H.; Lai, K.-Y.; Pramudityo, Chitosan Microneedle Patches for Sustained Transdermal Delivery of Macromolecules., Biomacromolecules. 13 (2012) 4022-4031.
[20] M.-C. Ling, M.-H.; Chen, Dissolving Polymer Microneedle Patches for Rapid and Efficient Transdermal Delivery of Insulin to Diabetic Rats, Acta Biomater. 9 (2013) 8952-8961.
[21] V. der Maaden, E. K., Sekerdag, P. Schipper, G. Kersten, W. Jiskoot, J. Bouwstra, Layer-by-Layer Assembly of Inactivated Poliovirus and N-Trimethyl Chitosan on PH-Sensitive Microneedles for Dermal Vaccination., Langmuir. 31 (2015) 8654-8660.
[22] National Center for Biotechnology Information. PubChem Database, Meloxicam, CID=54677470, (n.d.). https://pubchem.ncbi.nlm.nih.gov/compound/Meloxicam (accessed Mar. 12, 2020).
[23] J. Brugnerotto, J. Lizardi, F. M. Goycoolea, J. Argulles-Monal, W.; Desbrieres, M. Rinaudo, An infrared investigation in relation with chitin and chitosan characterization, Polymer (Guildf). 42 (2001) 3569-3580.
[24] M. Zheng, B. Han, Y. Yang, W. Liu, Synthesis, characterization and biological safety of O-carboxymethyl chitosan used to treat Sarcoma 180 tumor, Carbohydr. Polym. 86 (2011) 231-238.
[25] X. Wang, R. Tang, Y. Zhang, Z. Yu, C. Qi, Preparation of a Novel Chitosan Based Biopolymer Dye and Application in Wood Dyeing, Polymers (Basel). 8 (2016) 338.
[26] D. A. Castilla-Casadiego, H. V. Ramos-Avilez, S. Herrera-Posada, B. Calcagno, L. Loyo, J. Shipmon, A. Acevedo, A. Quintana, J. Almodovar, Engineering of a Stable Collagen Nanofibrous Scaffold with Tunable Fiber Diameter, Alignment, and Mechanical Properties, Macromol. Mater. Eng. 301 (2016) 1064-1075.
[27] D. A. Castilla-Casadiego, M. Maldonado, P. Sundaram, J. Almodovar, “Green” electrospinning of a collagen/hydroxyapatite composite nanofibrous scaffold, MRS Commun. 6 (2016) 402-407.
[28] M. A. Saleem, S. Bala, A. A. Liyakat, Effect of Different Carriers on in vitro Permeation of Meloxicam through Rat Skin, Indian J. Pharm. Sci. 72 (2010) 710-718.

We claim:

1. A microneedle array comprising a substrate and a plurality of microneedles extending therefrom, wherein the microneedles comprise a biodegradable polymer and an effective amount of an analgesic or an anti-inflammatory therapeutic agent and wherein the microneedles have a height configured to pierce the stratum corneum.

2. The microneedle array of claim 1, wherein the therapeutic agent is meloxicam.

3. The microneedle array of claim 1, wherein the biodegradable polymer is chitosan.

4. The microneedle array of claim 1, wherein the microneedle array comprises chitosan and 50-125 mg meloxicam per 64 mm².

5. The microneedle array of claim 1, wherein the microneedles have a height of 50-900 micron.

6. A method for treating a subject for pain or for pain management, the method comprising administering the microneedle array according to claim 1 to the epidermis of the subject, thereby piercing the stratum corneum of the subject.

7. The method of claim 6, wherein microneedle array sustainably releases the therapeutic agent for at least one week.

8. The method of claim 6, wherein the subject is a bovine.

9. The method of claim 6, wherein the microneedle array is administered by piercing the stratum corneum of the subject's ear

10. The method of claim 6, wherein the therapeutic agent is meloxicam.

11. The method of claim 6, wherein the biodegradable polymer is chitosan.

12. The method of claim 6, wherein the microneedle array comprises chitosan and 50-125 mg meloxicam per 64 mm².

13. A method for preparing a microneedle array, the method comprising:
applying a microneedle composition, the microneedle composition comprising a biodegradable polymer and an effective amount of an analgesic or an anti-inflammatory therapeutic agent to a mold, wherein the mold comprises a plurality recesses configured to prepare a plurality of microneedles;
distributing the microneedle composition within the plurality of recesses;
setting the microneedle array; and
releasing the microneedle array from the mold.

14. The method of claim 13 further comprising applying a substrate composition after setting the microneedle array.

15. The method of claim 13 further comprising applying a substrate composition prior to setting the microneedle array.

16. The method of claim 13, wherein the therapeutic agent is meloxicam.

17. The method of claim 13, wherein the biodegradable polymer is chitosan.

18. The method of claim 13, wherein the microneedle array comprises chitosan and 50-125 mg meloxicam per 64 mm².

19. The method of claim 13, wherein applying the microneedle composition and distributing the microneedle composition is repeated before setting the microneedle array.

20. The method of claim 13, wherein setting the microneedle array comprises drying the composition or heating the composition to an effective setting temperature for an effective setting time.
